(19) United States
(12) Patent Application Publication
(10) Pub. No.: US 2026/0216036 A1
(43) Pub. Date: Jul. 30, 2026

(54) COMPOUNDS FOR USE AS WETTING AGENTS AND/OR DISPERSING AGENTS

(71) Applicant: CLARIANT INTERNATIONAL LTD, Muttenz (CH)

(72) Inventors: Gundula STARKULLA, North Salem, NY (US); Borbala BEDE, Regensburg (DE); Sidonie VOLLRATH, Frankfurt am Main (DE); Carolina SIEDBUERGER, Frankfurt am Main (DE); Laura DREILING, Frankfurt am Main (DE)

(73) Assignee: CLARIANT INTERNATIONAL LTD, Muttenz (CH)

(21) Appl. No.: 19/145,246

(22) PCT Filed: Feb. 15, 2024
(86) PCT No.: PCT/EP2024/053892

§ 371 Date: Jul. 2, 2025

(30) Foreign Application Priority Data
Feb. 28, 2023 (EP) 23159263.5

(51) Int. Cl. A61K 8/60; A61K 8/29; A61K 8/35; A61K 8/37; A61K 8/40; A61Q 1/02; A61Q 1/06; A61Q 17/04

(52) U.S. Cl. CPC A61K 8/602; A61K 8/29; A61K 8/35; A61K 8/37; A61K 8/40; A61Q 1/02; A61Q 1/06; A61Q 17/04; A61K 2800/43

(57) ABSTRACT

The present invention relates to the use of compound X selected from glyceryl ethers, sorbitan esters, isosorbide esters, and compounds of Formula (I)

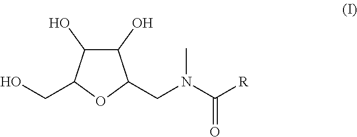

wherein R is selected from saturated hydrocarbon chains having 5 to 23 carbon atoms, unsaturated hydrocarbon chains having 5 to 23 carbon atoms, and mixtures thereof, as a wetting agent and/or dispersing agent.

The present invention relates to the use of certain compounds as wetting agents and/or dispersing agents.

Personal care is important for hygiene, well-being and beauty. Insoluble solids, for example pigments or UV filters, play a key functional role in products such as color cosmetics, moisturizers or sunscreens. They may also be used in other skin care products or in hair care products.

The effectiveness of the incorporation of such solids into a liquid matrix is often a key determinant in both the manufacturing process and the efficacy of the final formulation. The initial wetting of the solid particles is a key part of the process. During the wetting phase, air adsorbed onto the solid surface is displaced, and the solids are entirely surrounded by the liquid. This step may be followed by a dispersion step, in which high shear mixing is used to physically reduce the primary particle size of the solid, or to break up any agglomerates remaining after the initial wetting. In some cosmetic formulations, it is common to use only low to moderate shear, and to rely on effective wetting to achieve a stable dispersion having the required properties, for example a pleasant skin feel and/or good spreading properties of the particles.

There is an ongoing need for wetting agents and dispersing agents that show excellent performance.

Furthermore, there is a general need for ingredients for cosmetic formulations that are based on natural and renewable materials or are derived therefrom. Indeed, consumers are conscious as to the source of the ingredients used in such formulations, and these consumers feel more comfortable using ingredients that are derived from natural and renewable materials.

It has now been found that certain compounds can be used as wetting agents and/or dispersing agents.

Accordingly, the present invention relates to the use of compound X selected from glyceryl ethers, sorbitan esters, isosorbide esters, and compounds of Formula (I)

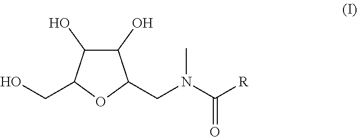

wherein R is selected from saturated hydrocarbon chains having 5 to 23 carbon atoms, unsaturated hydrocarbon chains having 5 to 23 carbon atoms, and mixtures thereof, as a wetting agent and/or dispersing agent.

The compounds used in the present invention have been known as preservative boosters and/or emollients or deoactives. They can now be additionally used as multifunctional ingredients, for example as preservative boosters and as wetting agents and/or dispersing agents. Advantageously, the compounds used in the present invention have a high renewable carbon content, i.e. they are sustainable.

According to the invention, compound X is selected from glyceryl ethers, sorbitan esters, isosorbide esters, and compounds of Formula (I)

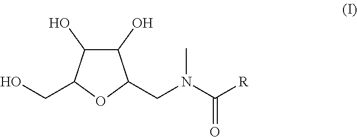

wherein R is selected from saturated hydrocarbon chains having 5 to 23 carbon atoms, unsaturated hydrocarbon chains having 5 to 23 carbon atoms, and mixtures thereof.

Preferably, compound X is selected from glyceryl ethers, sorbitan esters, and isosorbide esters. Particularly preferably, compound X is selected from glyceryl ethers.

In one embodiment, compound X is selected from glyceryl ethers.

Preferably, the glyceryl ethers are mono- or diethers of glycerin and one or more C6-C20 fatty alcohols. More preferably, the glyceryl ethers are mono- or diethers of glycerin and one or more C8-C14 fatty alcohols. Particularly preferably, the glyceryl ethers are monoethers of glycerin and one or more C8 fatty alcohols.

Preferably, the glyceryl ethers are selected from ethylhexylglycerin, methylheptylglycerin, caprylyl glyceryl ether, and mixtures thereof. More preferably, the glyceryl ethers are selected from ethylhexylglycerin, caprylyl glyceryl ether, and mixtures thereof. A particularly preferred glyceryl ether is caprylyl glyceryl ether.

In one embodiment, compound X is selected from sorbitan esters and isosorbide esters.

In one embodiment, compound X is selected from sorbitan esters.

Preferably, the sorbitan esters are mono-, di- or triesters of sorbitan and one or more C6-C20 fatty acids. More preferably, the sorbitan esters are mono- or diesters of sorbitan and one or more C8-C14 fatty acids. Particularly preferably, the sorbitan esters are mono- or diesters of sorbitan and caprylic acid.

Preferably, the sorbitan esters are selected from sorbitan caprylate, sorbitan stearate, sorbitan olivate, sorbitan oleate, sorbitan caprate, sorbitan laurate, sorbitan myristate, sorbitan caproate, and mixtures thereof. Particularly preferably, the sorbitan ester is sorbitan caprylate.

In one embodiment, compound X is selected from isosorbide esters.

Preferably, the isosorbide esters are mono- or diesters of isosorbide and one or more C6-C20 fatty acids. More preferably, the isosorbide esters are mono- or diesters of isosorbide and one or more C8-C14 fatty acids. Particularly preferably, the isosorbide esters are mono- or diesters of isosorbide and caprylic acid.

Preferably, the isosorbide esters are selected from isosorbide caprylate, isosorbide stearate, isosorbide olivate, isosorbide oleate, isosorbide caprate, isosorbide laurate, isosorbide myristate, isosorbide caproate, and mixtures thereof. Particularly preferably, the isosorbide ester is isosorbide caprylate.

In one embodiment, compound X is selected from compounds of Formula (I)

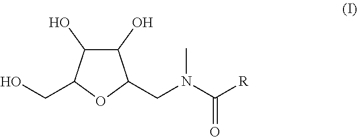

wherein R is selected from saturated hydrocarbon chains having 5 to 23 carbon atoms, unsaturated hydrocarbon chains having 5 to 23 carbon atoms, and mixtures thereof.

Preferably, R in Formula (I) is selected from saturated hydrocarbon chains having 5 to 17 carbon atoms, unsaturated hydrocarbon chains having 5 to 17 carbon atoms, and mixtures thereof.

More preferably, R in Formula (I) is selected from saturated hydrocarbon chains having 5 to 13 carbon atoms, unsaturated hydrocarbon chains having 5 to 13 carbon atoms, and mixtures thereof.

Particularly preferably, R in Formula (I) is selected from saturated hydrocarbon chains having 7 to 9 carbon atoms, unsaturated hydrocarbon chains having 7 to 9 carbon atoms, and mixtures thereof.

In at least one embodiment, R in Formula (I) is selected from —(CH₂)₆CH₃, —(CH₂)₈CH₃, and mixtures thereof; or R in Formula (I) is selected from —(CH₂)₁₀CH₃, —(CH₂)₁₂CH₃, and mixtures thereof; or R in Formula (I) is selected from —(CH₂)₇CH═CH₂, —(CH₂)₇CH═CHCH₂CH₃, and mixtures thereof; or the R—C═O residue in Formula (I) is derived from coconut fatty acids; or the R—C═O residue in Formula (I) is derived from soybean fatty acids.

In at least one embodiment, R in Formula (I) is selected from —(CH₂)₆CH₃, —(CH₂)₈CH₃, and mixtures thereof. In at least one embodiment, R in Formula (I) is selected from —(CH₂)₁₀CH₃, —(CH₂)₁₂CH₃, and mixtures thereof. In at least one embodiment, R in Formula (I) is selected from —(CH₂)₇CH═CH₂, —(CH₂)₇CH═CHCH₂CH₃, and mixtures thereof. In at least one embodiment, the R—C═O residue in Formula (I) is derived from coconut fatty acids. In at least one embodiment, the R—C═O residue in Formula (I) is derived from soybean fatty acids.

As used herein, the expression “the R—C═O residue in Formula (I) is derived from coconut fatty acids” preferably means that the carbon chain length distribution in the R—C═O residue in Formula (I) corresponds to the carbon chain length distribution of the fatty acids (e.g. bound as triglycerides) in coconut oil.

As used herein, the expression “the R—C═O residue in Formula (I) is derived from soybean fatty acids” preferably means that the carbon chain length distribution in the R—C═O residue in Formula (I) corresponds to the carbon chain length distribution of the fatty acids (e.g. bound as triglycerides) in soybean oil.

Preferably, the weight ratio of compounds according to Formula (I) wherein R is —(CH₂)₆CH₃ and compounds according to Formula (I) wherein R is —(CH₂)₈CH₃ is from 1:9 to 9:1, preferably from 3:7 to 7:3.

Preferably, the weight ratio of compounds according to Formula (I) wherein R is —(CH₂)₁₀CH₃ and compounds according to Formula (I) wherein R is —(CH₂)₁₂CH₃ is from 1:9 to 9:1, preferably from 3:7 to 7:3.

Preferably, the weight ratio of compounds according to Formula (I) wherein R is —(CH₂)₇CH═CH₂ and compounds according to Formula (I) wherein R is —(CH₂)₇CH═CHCH₂CH₃ is from 1:9 to 9:1, preferably from 3:7 to 7:3.

Compounds of Formula (I) are described in, e.g., WO 2018/002100 and EP application number EP21200587.0.

In a preferred embodiment, compound X is selected from caprylyl glyceryl ether, ethylhexylglycerin, sorbitan caprylate, isosorbide caprylate, and mixtures thereof. In a more preferred embodiment, compound X is selected from caprylyl glyceryl ether and ethylhexylglycerin. In a particularly preferred embodiment, compound X is caprylyl glyceryl ether.

According to the invention, compound X is used as a wetting agent and/or dispersing agent. In a preferred embodiment, compound X is used as a wetting agent. In a preferred embodiment, compound X is used as a dispersing agent. In a preferred embodiment, compound X is used as a wetting agent and a dispersing agent.

Preferably, the wetting agent is a pigment wetting agent, wetting agent for sun protection agents, or wetting agent for particulate substances. More preferably, the wetting agent is a pigment wetting agent or a wetting agent for sun protection agents.

Preferably, the dispersing agent is a pigment dispersing agent, dispersing agent for sun protection agents, or dispersing agent for particulate substances. More preferably, the dispersing agent is a pigment dispersing agent or a dispersing agent for sun protection agents.

Preferably, the pigment is an inorganic pigment. Preferred pigments are described further below. Preferably, the sun protection agent is an inorganic sun protection agent. Preferably, the sun protection agent is a UV filter. Preferably, the UV filter is an inorganic UV filter. Particularly preferably, the sun protection agent is an inorganic UV filter. Preferred sun protection agents are described further below.

In a preferred embodiment, compound X is used in a cosmetic formulation. Preferably, the cosmetic formulation is a skin care formulation or hair care formulation. Particularly preferably, the cosmetic formulation is a skin care formulation. Also preferably, the cosmetic formulation is a hair care formulation. In one embodiment, compound X is used in a home care formulation.

In preferred embodiments, the cosmetic formulation is selected from the group consisting of shampoo, body wash, facial cleanser, face mask, bubble bath, intimate wash, bath oil, cleansing milk, micellar water, make-up remover, hair mask, liquid soap, shaving soap, shaving foam, cleansing foam, day cream, anti-ageing cream, body milk, body lotion, body mousse, face serum, eye cream, sunscreen lotion, sun cream, face cream, after-shave lotion, pre-shaving cream, depilatory cream, skin-whitening gel, self-tanning cream, anti-acne gel, mascara, foundation, primer, concealer, blush, bronzer, blemish balm (bb) cream, eyeliner, night cream, eye brow gel, highlighter, lip stain, hand sanitizer, hair oil, nail varnish remover, conditioner, hair styling gel, hair styling cream, anti-frizz serum, scalp treatment, hair colorant, split end fluid, deodorant, antiperspirant, baby cream, insect repellent, hand cream, sunscreen gel, foot cream, exfoliator, body scrub, cellulite treatment, bar soap, cuticle cream, lip balm, hair treatment, eye shadow, bath additive, body mist, mouthwash, toothpaste, lubricating gel, moisturizer, serum, toner, aqua sorbet, cream gel, styling mousse, dry shampoo, lip stick, lip gloss, hydro-alcoholic gel, body oil, shower milk, illuminator, lip crayon, hair spray, combing cream, and sunblock.

In preferred embodiments, the cosmetic formulation is selected from the group consisting of body wash, facial cleanser, cleansing mask, bubble bath, bath oil, cleansing milk, micellar water, make-up remover, soaps, shaving soap, shaving foam, cleansing foam, face mask, intimate wash, micellar water, liquid soap, day cream, anti-aging cream, body milk, body lotion, body mousse, face serum, eye cream, sunscreen lotion, sun cream, face cream, after-shave lotion, pre-shaving cream, depilatory cream, skin-whitening gel, self-tanning cream, anti-acne gel, mascara, foundation, primer, concealer, blush, bronzer, blemish balm (bb) cream, eyeliner, night cream, eye brow gel, highlighter, lip stain, hand sanitizer, nail varnish remover, skin conditioner, split end fluid, deodorant, antiperspirant, baby cream, insect repellent, hand cream, sunscreen gel, foot cream, exfoliator, body scrub, cellulite treatment, bar soap, nail cuticle cream, lip balm, eye shadow, bath additive, body mist, lubricating gel, moisturizer, serum, toner, aqua sorbet, cream gel, lip stick, lip gloss, hydro-alcoholic gel, body oil, shower milk, illuminator, lip crayon, and sunblock.

Preferably, compound X is used in the cosmetic formulation in an amount of from 0.1 to 10 wt.-%, more preferably from 0.2 to 5 wt.-%, even more preferably from 0.3 to 3 wt.-%, particularly preferably from 0.5 to 2 wt.-%, based on the total weight of the cosmetic formulation.

In preferred embodiments, the cosmetic formulation comprises a pigment, sun protection agent, or particulate substance. In more preferred embodiments, the cosmetic formulation comprises a pigment or sun protection agent.

Preferably, the pigment is an inorganic pigment. Preferably, the sun protection agent is an inorganic sun protection agent. Preferably, the sun protection agent is a UV filter. Preferably, the UV filter is an inorganic UV filter. Particularly preferably, the sun protection agent is an inorganic UV filter.

In a preferred embodiment, the cosmetic formulation comprises a pigment. These may be colored pigments which impart color effects to the product mass or to the skin or hair, or they may be luster effect pigments which impart luster effects to the product mass or to the skin or hair. The color or luster effects on the skin are preferably temporary, i.e. they last until the next skin wash and can be removed by washing the skin with customary body or face cleansers, body or face washes etc. or by using a make-up remover, micellar water or cleansing wipes. The color or luster effects on the hair may be temporary. In at least one embodiment, the particle size of the pigment is from 1 micron to 200 micron, preferably from 3 micron to 150 micron, more preferably from 10 micron to 100 micron. The pigments are colorants which are virtually insoluble in the application medium and may be inorganic or organic. Inorganic-organic mixed pigments are also possible. Preference is given to inorganic pigments. The advantage of inorganic pigments is their excellent resistance to light, weather and temperature. The inorganic pigments may be of natural origin. In at least one embodiment, the inorganic pigment is selected from the group consisting of chalk, ochre, umber, green earth, burnt sienna, graphite, and combinations thereof. The pigments may be white pigments, such as, for example, titanium dioxide or zinc oxide, black pigments, such as, for example, iron oxide black, colored pigments, such as, for example, ultramarine, iron oxide red or iron oxide yellow, luster pigments, metal effect pigments, pearlescent pigments, or fluorescent or phosphorescent pigments. In at least one embodiment, the pigment is selected from the group consisting of metal oxides, hydroxides and oxide hydrates, mixed phase pigments, sulfur-containing silicates, metal sulfides, complex metal cyanides, metal sulfates, chromates and molybdates, and the metals themselves (bronze, silver, gold pigments), and combinations thereof. In at least one embodiment, the pigment is selected from the group consisting of titanium dioxide (CI 77891), black iron oxide (CI 77499), yellow iron oxide (CI 77492), red and brown iron oxide (CI 77491), manganese violet (CI 77742), ultramarine (sodium aluminum sulfosilicates, CI 77007, Pigment Blue 29), chromium oxide hydrate (CI 77289), Prussian blue (ferric ferrocyanide, CI 77510), carmine (cochineal), and combinations thereof. In at least one embodiment, the pigment is selected from the group consisting of pearlescent and colored pigments which consist of either single crystals like mica, silica, bismuth oxychloride, boron nitride or titanium dioxide or which have a layer-substrate structure based on mica, aluminum, aluminum oxide, titanium dioxide, silicium dioxide, silicates (e.g. calcium aluminum borosilicate, calcium sodium borosilicate, magnesium aluminum silicate or sodium magnesium fluorosilicate) which are coated with a metal oxide (e.g. iron oxide, chromium oxide, tin oxide, titanium dioxide) or a metal oxychloride, such as bismuth oxychloride, or a metal hydroxide, such as aluminum hydroxide, and optionally further color-imparting substances, such as Prussian blue, ultramarine, or carmine and where the color can be determined by varying the layer thickness. The pearlescent effect can be controlled both by means of the particle size and by means of the particle size distribution of the pigment population. Suitable particle size distributions are e.g. in the range 2-50 μm, 5-25 μm, 5-40 μm, 5-60 μm, 5-95 μm, 5-100 μm, 10-60 μm, 10-100 μm, 10-125 μm, 20-100 μm, 20-150 μm, or <15 μm. A wider particle size distribution, e.g. of 20-150 μm, produces glittering effects, whereas a narrower particle size distribution of <15 μm gives a uniform silky appearance. In at least one embodiment, the pigment is selected from the group consisting of titanium dioxide (CI 77891), black iron oxide (CI 77499), yellow iron oxide (CI 77492), red and brown iron oxide (CI 77491), mica, silica, bismuth oxychloride, and combinations thereof. In at least one embodiment, the pigment is selected from the group consisting of organic pigments, such as sepia, gamboge, bone charcoal, Cassel brown, indigo, chlorophyll or other plant pigments. In at least one embodiment, the pigment is selected from the group consisting of synthetic organic pigments, such as azo pigments, anthraquinoids, indigoids, dioxazine, quinacridone, phthalocyanine, isoindolinone, perylene, perinone, metal complex, alkali blue and diketopyrrolopyrrole pigments. In at least one embodiment, the cosmetic formulation comprises from 0.1 to 50 wt.-%, preferably from 0.5 to 40 wt.-%, more preferably from 1.0 to 30 wt.-%, particularly preferably from 3.0 to 20 wt.-% of a pigment, based on the total weight of the cosmetic formulation. In at least one embodiment, the cosmetic formulation comprises from 0.01 to 40 wt.-%, preferably from 0.1 to 30 wt.-%, more preferably from 0.5 to 20 wt.-%, particularly preferably from 1 to 10 wt.-% of a pigment, based on the total weight of the cosmetic formulation. In at least one embodiment, the cosmetic formulation comprises at least 3 wt.-%, preferably at least 10 wt.-%, more preferably at least 20 wt.-%, particularly preferably at least 30 wt.-% of a pigment, based on the total weight of the cosmetic formulation.

In a preferred embodiment, the cosmetic formulation comprises a sun protection agent. In preferred embodiments, the sun protection agent is selected from titanium dioxide, mica-titanium oxide, iron oxides, mica-iron oxide, zinc oxide, silicon oxides, ultramarine blue and chromium oxides. The cosmetic formulations can, for example, comprise titanium dioxide, mica-titanium oxide, iron oxides, mica-iron oxide, zinc oxide, silicon oxides, ultramarine blue or chromium oxides as pigments and as sun protection agents. In at least one embodiment, the cosmetic formulation comprises from 0.01 to 30 wt.-%, preferably from 0.05 to 20 wt.-%, more preferably from 0.1 to 15 wt.-%, particularly preferably from 1 to 10 wt.-% of a sun protection agent, based on the total weight of the cosmetic formulation. In at least one embodiment, the cosmetic formulation comprises from 0.01 to 40 wt.-%, preferably from 0.05 to 30 wt.-%, more preferably from 0.1 to 20 wt.-%, particularly preferably from 1 to 15 wt.-% of a sun protection agent, based on the total weight of the cosmetic formulation. In at least one embodiment, the cosmetic formulation comprises at least 2 wt.-%, preferably at least 3 wt.-%, more preferably at least 5 wt.-%, particularly preferably at least 10 wt.-% of a sun protection agent, based on the total weight of the cosmetic formulation.

In a preferred embodiment, the cosmetic formulation comprises a particulate substance. Suitable particulate substances are, for example, substances which are solid at room temperature (25° C.) and are in the form of particles. In at least one embodiment, the particulate substance is selected from the group consisting of silica, silicates, aluminates, clay earths, mica, kaolin, montmorillonite, insoluble salts, in particular insoluble inorganic metal salts, metal oxides, e.g. titanium dioxide, zinc oxide, minerals and insoluble polymer particles. In at least one embodiment, the particulate substance is selected from the group consisting of silica (silica gel, silicon dioxide) and metal salts, in particular inorganic metal salts. Metal salts are, for example, alkali metal or alkaline earth metal halides, such as sodium chloride or potassium chloride, or alkali metal or alkaline earth metal sulfates, such as sodium sulfate or magnesium sulfate. In at least one embodiment, the cosmetic formulation comprises from 0.01 to 10 wt.-%, preferably from 0.05 to 5 wt.-%, particularly preferably from 0.1 to 3 wt.-% of a particulate substance, based on the total weight of the cosmetic formulation.

In a preferred embodiment, the cosmetic formulation comprises an emollient. In at least one embodiment, the emollient is selected from triglycerides, esters, oils, butters, waxes, hydrocarbons, silicones, and mixtures thereof. In at least one embodiment, the emollient is selected from caprylic/capric triglyceride, C12-C15 alkyl benzoate, castor oil, hydrogenated castor oil, lauryl/myristyl polyricinoleate, polyglyceryl-3-polyricinoleate, glyceryl oleate, glyceryl rosinate, coco-caprylate/caprate, hydrogenated ethylhexyl olivate, olive oil unsaponifiables, hydrogenated olive oil unsaponifiables, Abyssinian oil, macadamia integrifolia/tetraphylla seed oil, cotton seed oil, grape seed oil, squalane, carnauba wax, beeswax, candelilla wax, shea butter, tucuma butter, cocoa seed butter, petrolatum, alkoxylated fatty alcohols, dimethicone, cyclomethicone, and mixtures thereof. In at least one embodiment, the cosmetic formulation comprises from 1 to 90 wt.-%, preferably from 5 to 50 wt.-%, particularly preferably from 5 to 30 wt.-% of an emollient, based on the total weight of the cosmetic formulation.

In a preferred embodiment, the cosmetic formulation is a leave-on skin care formulation. In a preferred embodiment, the cosmetic formulation is a color cosmetic formulation, a moisturizer or a sunscreen. The moisturizer is preferably a skin moisturizer. In a preferred embodiment, the cosmetic formulation is a moisturizer, preferably a skin moisturizer. In a preferred embodiment, the cosmetic formulation is a color cosmetic formulation or a sunscreen. In a particularly preferred embodiment, the cosmetic formulation is a color cosmetic formulation. In a particularly preferred embodiment, the cosmetic formulation is a sunscreen.

The present invention also relates to a combination comprising
(A) compound X selected from glyceryl ethers, sorbitan esters, isosorbide esters, and compounds of Formula (I)

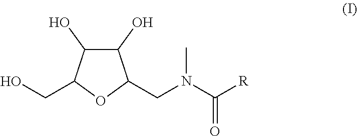

wherein R is selected from saturated hydrocarbon chains having 5 to 23 carbon atoms, unsaturated hydrocarbon chains having 5 to 23 carbon atoms, and mixtures thereof; and
(B) an emollient.

Preferred compounds X are described further above. Preferred emollients are described further above.

The following examples are intended to illustrate the invention without restricting it thereto.

### EXAMPLES

If not stated otherwise, % refers to wt.-%.

### Materials

CCT: Caprylic/Capric Triglyceride. CCT is a known emollient.

Velsan© CGE is commercially available from Clariant. Chemical name: Caprylyl Glyceryl Ether.

Velsan© EHG is commercially available from Clariant. Chemical name: Ethylhexylglycerin.

Velsan© SC is commercially available from Clariant. Chemical name: Sorbitan Caprylate.

### Example 1: Pigment Wetting

The viscosity of a pigment dispersion in oil is negatively correlated to the dispersion efficacy of the carrier material. Therefore, the decreased viscosities confirm the homogeneity of the dispersion. To calculate the mentioned viscosity difference ratio between the pure carrier material without added pigment and the pigment dispersion, following formula will be used:

Viscosity difference ratio=(viscosity value after pigment dispersion−viscosity value before pigment dispersion)/(viscosity value before pigment dispersion)

The lower the viscosity difference rate the better the pigment wetting.

Mixtures of 64% emollient (CCT) plus 1% preservative booster (Velsan CGE or Velsan EHG) with 35% pigment (red iron oxide, yellow iron oxide or Titanium Dioxide) were homogenized (2 min Ultra-Turrax @ 13000 rpm) and analyzed (Rheometer: HAAKE Mars III with Plate P35 Ti L-14048 (plate-and-plate method, gap 0.200 mm), average of 3 measurements: 30.0 s @ 20.0° C. as set temperature and sheer rate of 10.0 1/s).

TABLE 1

| | | | Viscosity |
|---|---|---|---|
| | | | difference |
| Example | Pigment | Sample | rate |
| 1a (Comparative) | Yellow | CCT | 463 |
| | (iron oxide) | | |
| 1b | Yellow | CCT + 1% Velsan EHG | 204 |
| | (iron oxide) | | |
| 1c | Yellow | CCT + 1% Velsan CGE | 118 |
| | (iron oxide) | | |
| 1d (Comparative) | Red | CCT | 32 |
| | (iron oxide) | | |
| 1e | Red | CCT + 1% Velsan EHG | 27 |
| | (iron oxide) | | |
| 1f | Red | CCT + 1% Velsan CGE | 11 |
| | (iron oxide) | | |
| 1g (Comparative) | White | CCT | 971 |
| | (Titanium | | |
| | Dioxide) | | |
| 1h | White | CCT + 1% Velsan CGE | 659 |
| | (Titanium | | |
| | Dioxide) | | |
| 1i | White | CCT + 1% Velsan EHG | 946 |
| | (Titanium | | |
| | Dioxide) | | |

Velsan OGE shows excellent wetting of pigments. These results show that Velsan OGE is a very useful additive for color cosmetic and mineral sun screen applications. Velsan EHG shows good results as well and is thus also suitable for such applications.

### Example 2: Particle Size

Mixtures of 64% emollient (CCT) plus 1% preservative booster (Velsan GE, Velsan EHG or Velsan SC) with 35% pigment (red iron oxide, yellow iron oxide or Titanium Dioxide) were homogenized (2 min Ultra-Turrax @ 13000 rpm) and analyzed using a grindometer (measurement (next day): average of 3 measurements with grindometer (0-100 μm, Thierry GmbH)).

TABLE 2

| | | | Particle |
|---|---|---|---|
| | | | size |
| Example | Pigment | Sample | [μm] |
| 2a (Comparative) | Yellow | CCT | 70 |
| | (iron oxide) | | |
| 2b | Yellow | CCT + 1% Velsan EHG | 50 |
| | (iron oxide) | | |
| 2c | Yellow | CCT + 1% Velsan SC | 30 |
| | (iron oxide) | | |
| 2d | Yellow | CCT + 1% Velsan CGE | 15 |
| | (iron oxide) | | |
| 2e (Comparative) | Red | CCT | 100 |
| | (iron oxide) | | |
| 2f | Red | CCT + 1% Velsan EHG | 95 |
| | (iron oxide) | | |
| 2g | Red | CCT + 1% Velsan CGE | 95 |
| | (iron oxide) | | |
| 2h (Comparative) | White | CCT | 100 |
| | (Titanium | | |
| | Dioxide) | | |
| 2i | White | CCT + 1% Velsan EHG | 97.5 |
| | (Titanium | | |
| | Dioxide) | | |
| 2j | White | CCT + 1% Velsan CGE | 95 |
| | (Titanium | | |
| | Dioxide) | | |

Velsan CGE shows excellent dispersion of pigments with lower average particle size compared to neat COT. These results show that Velsan CGE is very useful for color cosmetic and mineral sun screen applications. Velsan EHG and Velsan SC show good results as well and are thus also suitable for such applications.

In conclusion, Caprylyl Glyceryl Ether, Ethylhexylglycerin or Sorbitan Caprylate cannot only be used as preservative boosters but also as wetting agents and/or dispersing agents for pigments or inorganic UV filters.

### Example 3: Cosmetic Formulation

A lipstick was prepared using the following ingredients:

| | | | Amount |
|---|---|---|---|
| | Tradename | INCI | (%) |
| A | Castor Oil | | 32.60 |
| | Plantasens ® | Lauryl-/Myristyl-Polyricinoleate and | 10.00 |
| | Pro LM | Glycerin | |
| | Plantasens ® Olive | Squalane | 3.10 |
| | Squalane | | |
| | Plantasens ® | Macadamia Integrifolia/Tetraphylla | 2.70 |
| | Macdamia | Seed Oil | |
| | Oil refined | | |
| | Plantasens ® Grape | Vitis Vinifera (Grape) Seed Oil | 1.20 |
| | Seed Oil refined | | |
| | Plantasens ® Shea | Butyrospermum Parkii (Shea) Butter | 5.75 |
| | Butter | | |
| | Plantasens ® Cocoa | Theobroma Cacao (Cocoa) Seed | 0.75 |
| | Butter | Butter | |
| | Plantasens ® | Hydrogenated Ethylhexyl Olivate | 4.00 |
| | Olive LD | (and) Hydrogenated Olive Oil | |
| | | Unsaponifiables | |
| | Plantasens ® | Polyglyceryl-2 Sesquiisostearate | 3.00 |
| | Emulsifier DGI | | |
| | Tocopheryl Acetate | Tocopheryl Acetate | 0.20 |
| | Carnauba Wax | Copernicia Cerifera (Carnauba) Wax | 4.50 |
| | Candelilla Wax | Euphorbia Cerifera (Candelilla) Wax | 4.50 |
| | Beeswax | Cera Alba (Bees) Wax | 4.50 |
| B | Eusolex ® T-2000 | Titanium Dioxide (and) Alumina | 8.00 |
| | | (and) Simethicone | |
| | Xirona ® Le Rouge | Iron Oxides (and) Silica | 8.50 |
| | Gemtone ® | Mica (and) Titanium Dioxide (and) | 4.00 |
| | Tan Opal | Iron Oxides | |
| | ECONA ™ N-5441S | Mica (and) Tin Oxide (and) Titanium | 2.50 |
| | Sappan Red | Dioxide (and) Caesalpinia Sappan | |
| | | Bark Extract | |
| C | Velsan ® CGE | Caprylyl Glyceryl Ether | 0.20 |

### Procedure:

I. Mix Components of A and heat to 8000.
II. Mix components of B.
III. Add II to I and stir with a propeller stirrer with a max. speed of approx. 200 rpm until pigments are fully wetted. Temperature needs to be above 6000.
IV. Add C to Ill and stir until homogenous.
V. Pour formulation in a lipstick mold and allow to cool down.

### Technical Data:

Viscosity (20° C., 20 rpm): solid
Appearance: red lipstick
Stability: Stable for 12 weeks @ RT, 40° C.

### Example 4: Cosmetic Formulation

A tinted day crème was prepared using the following ingredients:

| | | | Weight |
|---|---|---|---|
| | Tradename | INCI | % |
| A | Water | Aqua | Ad 100 |
| | Glycerin 85% | Glycerin | 2.00 |
| B | Hostaphat KL 340D | Trilaureth-4 Phosphate | 2.50 |
| | Hostacerin DGI | Polyglyceryl-2 Sesquiisostearate | 1.00 |
| | Cetearyl Alcohol | Cetearyl Alcohol | 1.50 |
| | Octocrylene | Octocrylene | 7.00 |
| | Butyl | Butyl Methoxydibenzoylmethane | 4.50 |
| | Methoxydibenzoylmethane | | |
| | Ethylhexyl Salicylate | Ethylhexyl Salicylate | 2.50 |
| | C12-15 Alkyl Benzoate | C12-15 Alkyl Benzoate | 7.00 |
| | Plantasens Olive LD | Hydrogenated Ethylhexyl Olivate | 4.00 |
| | | (and) Hydrogenated Olive Oil | |
| | | Unsaponifiables | |
| | Plantasens Abyssinian Oil | Crambe Abyssinica Seed Oil | 2.00 |
| | Xiameter PMX-200 | Dimethicone | 4.00 |
| | Silicone Fluid 50 cs | | |
| C | Titanium Dioxide | Titanium Dioxide | 2.50 |
| | Chroma-Lite Black | Mica (and) Bismuth Oxychloride | 0.05 |
| | | (and) Iron Oxides | |
| | Chroma-Lite Red | Mica (and) Bismuth Oxychloride | 0.20 |
| | | (and) Iron Oxides | |
| | Chroma-Lite Yellow | Mica (and) Bismuth Oxychloride | 0.60 |
| | | (and) Iron Oxides | |
| | Butylene Glycol | Butylene Glycol | 4.00 |
| D | Aristoflex Velvet | Polyacrylate Crosspolymer-11 | 1.00 |
| E | Tocopherol Acetate | Tocopherol Acetate | 1.00 |
| | Sodium Hyaluronate | Sodium Hyaluronate | 0.05 |
| | Velsan CGE | Caprylyl Glyceryl Ether | 0.50 |
| | Nipaguard SCA | Sorbitan Caprylate (and) | 1.20 |
| | | Benzylalcohol | |
| F | Citric Acid/Sodium | Citric Acid/Sodium Hydroxide | qs |
| | Hydroxide | | |

### Procedure:

I. Mix the components of phase A and heat to 80° C.
II. Mix the components of phase B and heat to 80° C.
III. Premix the components of phase C separately
IV. At temperature, add III and phase D to II, while homogenizing with Ultraturrax
V. Add IV to I while using Ultraturrax. Then switch to finger stirrer and stir until 35-40° C.
VI. Add ingredients of phase E to V and stir until RT
VII. Adjust pH to 5.3-5.8

### Formulation Details/Stability:

pH: 5.0-5.5
Viscosity: 18000 m Pas
T Appearance: light beige cream
Stability: stable at RT, 40° C. and 0° C. for 8 weeks
Challenge test DIN ISO 11930: A pass

### Example 5: Cosmetic Formulation

A mineral sun defense SPF 20 (SPF calculated) was prepared using the following ingredients:

| | | | Weight |
|---|---|---|---|
| | Tradename | INCI | % |
| A | Water | Aqua | Ad 100 |
| | Glycerin 85% | Glycerin | 4.70% |
| | Xanthan Gum | Xanthan Gum | 0.10% |
| | Magnesium Sulfate | Magnesium Sulfate | 0.50% |
| | Heptahydrate | | |
| B | Lanette O | Cetearyl Alcohol | 2.00% |
| | Magnesium Stearate | Magnesium Stearate | 1.00% |
| | Plantasens Shea Butter | Butyrospermum Parkii | 2.00% |
| | | (Shea) Butter | |
| | Plantasens VP R15 | Ricinus Communis (Castor) Seed | 2.00% |
| | | Oil (and) Hydrogenated Castor | |
| | | Oil (and) Copernicia Cerifera | |
| | | (Carnauba) Wax | |
| | Plantasens Emulsifier CP5 | Glyceryl Oleate (and) | 4.00% |
| | | Polyglyceryl-3 Polyricinoleate | |
| | | (and) Olea Europaea (Olive) Oil | |
| | | Unsaponifiables | |
| | C12-15 Alkyl Benzoate | C12-15 Alkyl Benzoate | 7.00% |
| | Plantasens Abyssinian Oil | Crambe Abyssinica Seed Oil | 3.00% |
| C | Eusolex T AVO | Titanium Dioxide (and) Silica | 15.00% |
| D | Velsan CGE | Caprylyl Glyceryl Ether | 0.5% |
| | Nipaguard SCP | Sorbitan Caprylate (and) | 1.00% |
| | | Phenoxyethanol | |
| E | HerbExMagnoSoother | Butylene Glycol (and) Magnolia | 1.00% |
| | | Kobus Bark Extract | |
| | Fragrance “Golden | Perfume | 0.30% |
| | Temptation” | | |

### Procedure:

I. Mix A until the gel is homogeneous. Adjust pH to 5.5, then heat to 85° C.
II. Mix B and heat to 85° C.
III. Add C to A
IV. Add II to III while stirring at high speed until homogeneous
V. Cool down under stirring and add D at 35° C. and E

### Formulation Details/Stability:

pH: 7.4
Appearance: white cream
Stability: stable at RT, 40° C. and 0° C. for 12 weeks
Challenge test DIN ISO 11930: A pass

1-14. (canceled)

15. A method of wetting and/or dispersing solid particles, the method comprising contacting compound X with the solid particles, wherein compound X is one or more selected from glyceryl ethers, sorbitan esters, isosorbide esters, and compounds of Formula (I) 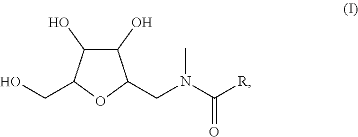
where R is selected from saturated hydrocarbon chains having from 5 to 23 carbon atoms and unsaturated hydrocarbon chains having from 5 to 23 carbon atoms.

16. The method of claim 15, wherein the solid particles are one or more selected from pigments, sun protection agents, and particulate substances.

17. The method of claim 15, wherein the solid particles are one or more selected from metals, metal oxides, metal hydroxides, metal oxide hydrates, mixed phase pigments, sulfur-containing silicates, metal sulfides, complex metal cyanides, metal sulfates, metal chromates, metal molybdates, pearlescent materials comprising single crystals, layer-substrate structures, organic pigments, silica, silicates, silicon oxides, inorganic metal salts, aluminates, clay earths, kaolin, montmorillonite, insoluble salts, minerals, and insoluble polymer particles.

18. The method of claim 15, wherein compound X is one or more glyceryl ethers, one or more sorbitan esters, one or more isosorbide esters, or mixtures of these.

19. The method of claim 15, wherein compound X is one or more selected from caprylyl glyceryl ether, ethylhexylglycerin, methylheptylglycerin, sorbitan caprylate, sorbitan stearate, sorbitan olivate, sorbitan oleate, sorbitan caprate, sorbitan laurate, sorbitan myristate, sorbitan caproate, isosorbide caprylate, isosorbide stearate, isosorbide olivate, isosorbide oleate, isosorbide caprate, isosorbide laurate, isosorbide myristate, and isosorbide caproate.

20. The method of claim 15, wherein compound X is one or more selected from caprylyl glyceryl ether, ethylhexylglycerin, sorbitan caprylate, and isosorbide caprylate.

21. The method of claim 15, wherein the method comprises contacting compound X with the solid particles in a cosmetic formulation.

22. The method of claim 21, wherein
the solid particles are pigments and are present in an amount of from 0.01 to 40 wt.-%, based on the total weight of the cosmetic formulation; and
compound X is present in an amount of from 0.1 to 10 wt.-%, based on the total weight of the cosmetic formulation.

23. The method of claim 21, wherein the cosmetic formulation further comprises one or more emollients selected from caprylic/capric triglyceride, C₁₂-C₁₅ alkyl benzoate, castor oil, hydrogenated castor oil, lauryl/myristyl polyricinoleate, polyglyceryl-3-polyricinoleate, glyceryl oleate, glyceryl rosinate, coco-caprylate/caprate, hydrogenated ethylhexyl olivate, olive oil unsaponifiables, hydrogenated olive oil unsaponifiables, Abyssinian oil, macadamia integrifolia/tetraphylla seed oil, cotton seed oil, grape seed oil, squalane, carnauba wax, beeswax, candelilla wax, shea butter, tucuma butter, cocoa seed butter, petrolatum, alkoxylated fatty alcohols, dimethicone, and cyclomethicone.

24. A cosmetic formulation comprising
solid particles; and
compound X, wherein compound X is one or more selected from glyceryl ethers, sorbitan esters, isosorbide esters, and compounds of Formula (I) 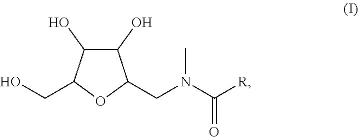
wherein R is selected from saturated hydrocarbon chains having from 5 to 23 carbon atoms and unsaturated hydrocarbon chains having from 5 to 23 carbon atoms.

25. The cosmetic formulation of claim 24, wherein the solid particles are one or more selected from pigments, sun protection agents, and particulate substances.

26. The cosmetic formulation of claim 24, wherein the solid particles are one or more selected from metals, metal oxides, metal hydroxides, metal oxide hydrates, mixed phase pigments, sulfur-containing silicates, metal sulfides, complex metal cyanides, metal sulfates, metal chromates, metal molybdates, pearlescent materials comprising single crystals, layer-substrate structures, organic pigments, silica, silicates, silicon oxides, inorganic metal salts, aluminates, clay earths, kaolin, montmorillonite, insoluble salts, minerals, and insoluble polymer particles.

27. The cosmetic formulation of claim 24, wherein
the solid particles are pigments and are present in an amount of from 0.01 to 40 wt.-%, based on the total weight of the cosmetic formulation; and
compound X is present in an amount of from 0.1 to 10 wt.-%, based on the total weight of the cosmetic formulation.

28. The cosmetic formulation of claim 24, wherein compound X is one or more glyceryl ethers, one or more sorbitan esters, one or more isosorbide esters, or mixtures of these.

29. The cosmetic formulation of claim 24, wherein compound X is one or more selected from caprylyl glyceryl ether, ethylhexylglycerin, methylheptylglycerin, sorbitan caprylate, sorbitan stearate, sorbitan olivate, sorbitan oleate, sorbitan caprate, sorbitan laurate, sorbitan myristate, sorbitan caproate, isosorbide caprylate, isosorbide stearate, isosorbide olivate, isosorbide oleate, isosorbide caprate, isosorbide laurate, isosorbide myristate, and isosorbide caproate.

30. The cosmetic formulation of claim 24, wherein compound X is one or more selected from caprylyl glyceryl ether, ethylhexylglycerin, sorbitan caprylate, and isosorbide caprylate.

31. The cosmetic formulation of claim 24, further comprising one or more emollients selected from caprylic/capric triglyceride, C₁₂-C₁₅ alkyl benzoate, castor oil, hydrogenated castor oil, lauryl/myristyl polyricinoleate, polyglyceryl-3-polyricinoleate, glyceryl oleate, glyceryl rosinate, coco-caprylate/caprate, hydrogenated ethylhexyl olivate, olive oil unsaponifiables, hydrogenated olive oil unsaponifiables, Abyssinian oil, macadamia integrifolia/tetraphylla seed oil, cotton seed oil, grape seed oil, squalane, carnauba wax, beeswax, candelilla wax, shea butter, tucuma butter, cocoa seed butter, petrolatum, alkoxylated fatty alcohols, dimethicone, and cyclomethicone.

32. A combination comprising
(A) compound X, wherein compound X is one or more selected from glyceryl ethers, sorbitan esters, isosorbide esters, and compounds of Formula (I) 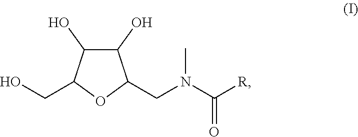
wherein R is selected from saturated hydrocarbon chains having from 5 to 23 carbon atoms and unsaturated hydrocarbon chains having from 5 to 23 carbon atoms; and
(B) an emollient.

33. The combination of claim 32, wherein
compound X is one or more selected from caprylyl glyceryl ether, ethylhexylglycerin, sorbitan caprylate, and isosorbide caprylate; and
the emollient is one or more selected from caprylic/capric triglyceride, C₁₂-C₁₅ alkyl benzoate, castor oil, hydrogenated castor oil, lauryl/myristyl polyricinoleate, polyglyceryl-3-polyricinoleate, glyceryl oleate, glyceryl rosinate, coco-caprylate/caprate, hydrogenated ethylhexyl olivate, olive oil unsaponifiables, hydrogenated olive oil unsaponifiables, Abyssinian oil, macadamia integrifolia/tetraphylla seed oil, cotton seed oil, grape seed oil, squalane, carnauba wax, beeswax, candelilla wax, shea butter, tucuma butter, cocoa seed butter, petrolatum, alkoxylated fatty alcohols, dimethicone, and cyclomethicone.
